(19) United States
(12) Patent Application Publication
(10) Pub. No.: US 2009/0318776 A1
(43) Pub. Date: Dec. 24, 2009

(54) PHYSICAL CONDITION MANAGEMENT SYSTEM

(75) Inventors: Hiroshi Toda, Aichi (JP); Masaki Ito, Shizuoka (JP); Takeyoshi Kaminishizono, Aichi (JP); Mitsuhiro Ando, Aichi (JP)

Correspondence Address: SUGHRUE MION, PLLC, 2100 PENNSYLVANIA AVENUE, N.W., SUITE 800, WASHINGTON, DC, 20037, US

(73) Assignee: AISIN SEIKI KABUSHIKI KAISHA, Kariya-shi, Aichi-ken (JP)

(21) Appl. No.: 12/442,318

(22) PCT Filed: Nov. 7, 2007
(86) PCT No.: PCT/JP2007/071613

§ 371 Date: Mar. 20, 2009

(51) Int. Cl. A61B 5/00; G08B 23/00

(52) U.S. Cl. 600/301; 340/575; 340/576

(57) ABSTRACT

A physical condition management system detects during-sleep biomedical information while the driver is sleeping and during-drive biomedical information while the driver drives a vehicle. A during-sleep physical condition information calculating section calculates during-sleep physical condition information from during-drive physical condition information and/or the during-drive biomedical information, and the during-sleep biomedical information. A during-drive physical condition information calculating section calculates the during-drive physical condition information from the during-sleep physical condition information and/or the during-sleep biomedical information, and the during-drive biomedical information. The sleep support operation is controlled according to the during-sleep physical condition information and the drive support operation is controlled according to the during-drive physical condition information. An information transmitting section transmits the during-sleep physical condition information and/or the during-sleep biomedical information to the during-drive physical condition information calculating section, and the during-drive physical condition information and/or the during-drive biomedical information to the during-sleep physical condition information calculating section.

### TECHNICAL FIELD

The present invention relates to a physical condition management system which obtains a biomedical information and manages a physical condition of a person.

### BACKGROUND ART

As an example of such physical condition management system, Japan Published Unexamined Patent Application H7-117595 (Patent Document 1) discloses a drowsy driving preventing apparatus. According to Patent Document 1, a blood volume of a driver is detected by a detecting means attached to an ear lobe of the driver. A varying pattern of the blood volume is produced from time to time based on the detected blood volume. Meanwhile, the driving preventing apparatus stores therein the varying pattern of the blood volume at the time of drowsy driving, which is compared to the produced varying pattern. When both patterns approximate each other, the driving preventing apparatus determines that the driver is in a drowsy state and provides the warning.

Japan Patent Publication 3588781 (Patent Document 2) discloses technology of a wakefulness control apparatus for a vehicle occupant. According to Patent Document 2, a cycle of waking rhythm of a person is measured, and waking stimulus and sleep inducing stimulus are provided based on the measured cycle of waking rhythm. When the vehicle is running, the wakefulness control apparatus presents the waking stimulus in synchronized with the cycle of waking rhythm. When the vehicle is not running, the wakefulness control apparatus stops presenting the waking stimulus to reduce wakefulness and presents the sleep inducing stimulus. The waking rhythm is measured by use of a telemeter in advance.

Japan Published Patent Application 2004-507308 (Patent Document 3 (Japanese translation of International Publication Gadget WO2002/017786) discloses technology related to a method for diagnosing a fitness of a driver and a device thereof. According to Patent Document 3, a stress amount of the driver is evaluated by combining physiological measured values obtained in the vehicle during travel, stationarily obtained or estimated health-relevant data of the driver and data indicating the stress exerted on the driver. Changes in a condition of the driver is weighted with the evaluated stress amount of the driver and interpreted. The stationary health-relevant data is measured by a bicycle ergometer or the like.

Patent Document 1: JP7-117595A (page 2, FIG. 2 and other)
Patent Document 2: JP3588781B2 (pages 2, 3 and other)
Patent Document 3: JP2004-507308A (pages 3, 4 and other)

### DISCLOSURE OF INVENTION

According to the drowsy driving preventing apparatus disclosed in Patent Document 1, the driver needs to attach the detecting means to his or her ear lobe. Consequently, the driver may find it troublesome to attach the detecting means or feel uncomfortable for keeping the detecting means attached. According to the wakefulness control apparatus disclosed in Patent Document 2, the cycle of waking rhythm needs to be measured. The waking rhythm is measured by use of the telemeter in advance, which may be troublesome to the user of the apparatus. According to the method for diagnosing the fitness of the driver disclosed in Patent Document 3, the driver needs to measure the stationary health-relevant data using a health appliance or the like, which may be troublesome to the driver. Further, as is also applied to Patent Document 2, accuracy of the determination may not improve when the waking rhythm or the stationarily measured health-relevant data is used in the evaluation because a physical condition of a person varies daily.

The present invention is made considering the problems described above and an object of the present invention is to provide a physical condition management system managing a physical condition of a person without bothering him or her based on his or her biomedical information obtained before and while in a vehicle.

To achieve the above-mentioned object, a physical condition management system of the present invention includes a during-sleep biomedical information detecting section detecting a during-sleep biomedical information during sleep, a sleep support operation control section controlling a sleep support operation supporting at least sleep induction based on a during-sleep physical condition information calculated by use of the during-sleep biomedical information, a during-drive biomedical information detecting section detecting a during-drive biomedical information while a user is in a vehicle, a drive support operation control section controlling a drive support operation supporting at least a driving operation of the vehicle based on a during-drive physical condition information calculated by use of the during-drive biomedical information, a during-sleep physical condition information calculating section calculating the during-sleep physical condition information based on either one or both of the during-drive physical condition information and the during-drive biomedical information, and the during-sleep biomedical information, a during-drive physical condition information calculating section calculating the during-drive physical condition information based on either one or both of the during-sleep physical condition information and the during-sleep biomedical information, and the during-drive biomedical information, and an information transmitting unit transmitting either one or both of the during-sleep physical condition information and the during-sleep biomedical information to the during-drive physical condition information calculating section, and transmitting either one or both of the during-drive physical condition information and the during-drive biomedical information to the during-sleep physical condition information calculating section.

According to this structure, the during-sleep biomedical information of the user detected during sleep is used as the biomedical information before getting in the vehicle. Since the during-sleep biomedical information is detected during sleep, the biomedical information of the user at rest is detected without bothering him or her. Either one or both of the during-sleep physical condition information calculated using the biomedical information and the during-sleep biomedical information is transmitted to the during-drive physical condition information calculating section via the information transmitting unit. Consequently, the during-drive physical condition information calculating section calculates the during-drive physical condition information based on the during-drive biomedical information obtained during driving, and the received during-sleep physical condition information and/or the received during-sleep biomedical information. The drive support operation control section controls the drive support operation based on the calculated during-drive physical condition information. That is, the drive support operation control section performs the drive support operation in response to the physical condition of the person to manage the physical condition during drive. Thus, the present invention provides the physical condition management system which manages the physical condition based on the biomedical information obtained before and while the person is in the vehicle without bothering him or her.

Further, either one or both of the during-drive physical condition information and the during-drive biomedical information during drive is transmitted to the during-sleep physical condition information calculating section via the information transmitting unit. The during-sleep physical condition information calculating section calculates the during-sleep physical condition information based on the during-sleep biomedical information and the information before sleep, that is, the during-drive physical condition information and/or the during-drive biomedical information. Then, the sleep support operation control section controls the sleep support operation based on the calculated during-sleep physical condition information. That is, the sleep support operation control section performs the sleep support operation in response to the physical condition of the person to manage the physical condition during sleep. It has been recently pointed out that an appropriate sleep plays an important role in managing the physical condition, and thus it is significantly meaningful, from a standpoint of managing the physical condition, to appropriately manage the physical condition during sleep and provide good sleep.

According to this structure, the management of the physical condition during drive and the management of the physical condition during sleep are closely linked. For example, when the during-sleep physical condition information corresponds to “good physical condition” but the during-drive physical condition calculated from the during-drive biomedical information corresponds to “exhausted”, the sleep support operation or the calculation of the during-sleep physical condition information might have been improperly performed. In such a case, transmitting the during-drive physical condition information to the during-sleep physical condition information calculating section leads to an improvement of the next calculation or the sleep support operation.

In addition to the above-mentioned structure, the physical condition management system of the present invention is characterized by the following structure. The during-drive physical condition information calculating section calculates the during-drive physical condition information including at least either one or both of a degree of concentration on driving and a degree of fatigue from driving based on the during-drive biomedical information, and the during-sleep physical condition information including at least either one or both of a quality of sleep and a degree of wakefulness at a time of awakening. The drive support operation control section operates the drive support operation including at least either one or both of an awakening operation and a rest induction operation based on the during-drive physical condition information.

According to this structure, the during-drive physical condition information calculating section calculates the physical condition information associated with drowsy driving including the degree of concentration on driving and the degree of fatigue from driving, in consideration of the physical condition information before the user gets in the vehicle. Thus, the awakening operation and the rest induction operation are performed by the drive support operation control section based on the highly accurate during-drive information, thereby enabling the appropriate management of the physical condition.

In addition to the above-mentioned structure, the physical condition management system of the present invention is characterized by the following structure. The during-sleep physical condition information calculating section calculates the during-sleep physical condition information including at least a required quality of sleep based on the during-sleep biomedical information, and the during-drive physical condition information including at least either one or both of a degree of excitement by driving and cumulative fatigue from driving. The sleep support operation control section operates the sleep support operation including at least either one or both of a sleep promotion operation and a sleep induction operation based on the during-sleep physical condition information.

According to this structure, the during-sleep physical condition information calculating section calculates the required sleep quality (depth of sleep, rhythm of sleep, hours of sleep and the like) which is important for managing the physical condition, in consideration of the physical condition information when the user is engaged in activities (before sleep). Thus, the sleep support operation including a promotion of sleep to relieve fatigue and a suppression of excitement to induce an early sleep are performed by the sleep support operation control section, thereby enabling the appropriate management of the physical condition. The sleep promotion operation is intended for relaxation brought by means of, for example, massage, music, lighting and the like. The sleep induction operation is intended for promoting sleep by, for example, applying minor vibrations (1/f fluctuation), supplying negative ions, and regulating temperature and humidity of a sleep environment.

The physical condition management system of the present invention is characterized by further including a driving operation detecting section detecting a driving operation of the vehicle and a running state detecting section detecting a running state of the vehicle. The during-drive physical condition information calculating section calculates the during-drive physical condition information based on the during-drive biomedical information, the during-sleep physical condition information, a driving operation information detected by the driving operation detecting section and a running state information detected by the running state detecting section.

According to this structure, the during-drive physical condition information is calculated considering the driving operation information and the running state information in addition to the during-drive biomedical information and the during-sleep physical condition information, thereby enabling even more appropriate management of the physical condition.

The physical condition management system of the present invention is characterized in that the information transmitting unit transmits the during-sleep physical condition information and the during-drive physical condition information by means of a communication medium or a rewritable storage medium.

Since the information transmitting unit includes the communication medium or the rewritable storage medium, the information is conveniently transmitted without bothering the user.

Other characteristics of the physical condition management system of the present invention is that the physical condition management system includes a during-sleep biomedical information detecting section detecting a during-sleep biomedical information during sleep, a during-sleep physical condition information calculating section calculating a during-sleep physical condition information based on the during-sleep biomedical information, a during-drive biomedical information detecting section detecting a during-drive biomedical information while the user is in a vehicle, a drive support operation control section controlling a drive support operation supporting at least a driving operation of the vehicle based on a during-drive physical condition information calculated by use of the during-drive biomedical information, a during-drive physical condition information calculating section calculating the during-drive physical condition information based on either one or both of the during-sleep physical condition information and the during-sleep biomedical information, and the during-drive biomedical information, and an information transmitting unit transmitting either one or both of the during-sleep physical condition information and the during-sleep biomedical information to the during-drive physical condition information calculating section.

According to this structure, the during-sleep biomedical information of the user detected during sleep is used as the biomedical information before getting in the vehicle. Since the during-sleep biomedical information is detected during sleep, the biomedical information of a user at rest is detected without bothering the user. Either one or both of the during-sleep physical condition information calculated using the biomedical information and the biomedical information is transmitted to the during-drive physical condition information calculating section via the information transmitting unit. Consequently, the during-drive physical condition information calculating section calculates the during-drive physical condition information based on the during-drive biomedical information obtained during driving, and the during-sleep physical condition information and/or the during-sleep biomedical information. The drive support operation control section controls the drive support operation based on the calculated during-drive physical condition information. That is, the drive support operation control section performs the drive support operation in response to the physical condition of the person to manage the physical condition during drive. Thus, the present invention provides the physical condition management system which manages the physical condition based on the biomedical information obtained before and while the person is in the vehicle without bothering him or her.

### BRIEF DESCRIPTION OF DRAWINGS

FIG. 1 is a block diagram schematically showing an example of a structure of a physical condition management system of the present invention;

FIG. 2 is a block diagram schematically showing an application example of the physical condition management system of the present invention;

FIG. 3 is a diagram explaining an example of a sleep system shown in FIG. 2;

FIG. 4 is a diagram explaining an example of a vehicle shown in FIG. 2;

FIG. 5 is a block diagram schematically showing another example of the structure of the physical condition management system of the present invention;

FIG. 6 is a state transition diagram of a physical condition management;

FIG. 7 is a diagram explaining an example of a state that information is shared;

FIG. 8 is a block diagram schematically showing another application example of the physical condition management system of the present information;

FIG. 9 is a block diagram schematically showing another application example of the physical condition management system of the present invention;

FIG. 10 a block diagram schematically showing another application example of the physical condition management system of the present invention;

FIG. 11 is a block diagram schematically showing another example of the structure of the physical condition management system of the present invention; and

FIG. 12 is a block diagram schematically showing another example of the structure of the physical condition management system of the present invention.

### MODE FOR CARRYING OUT THE INVENTION

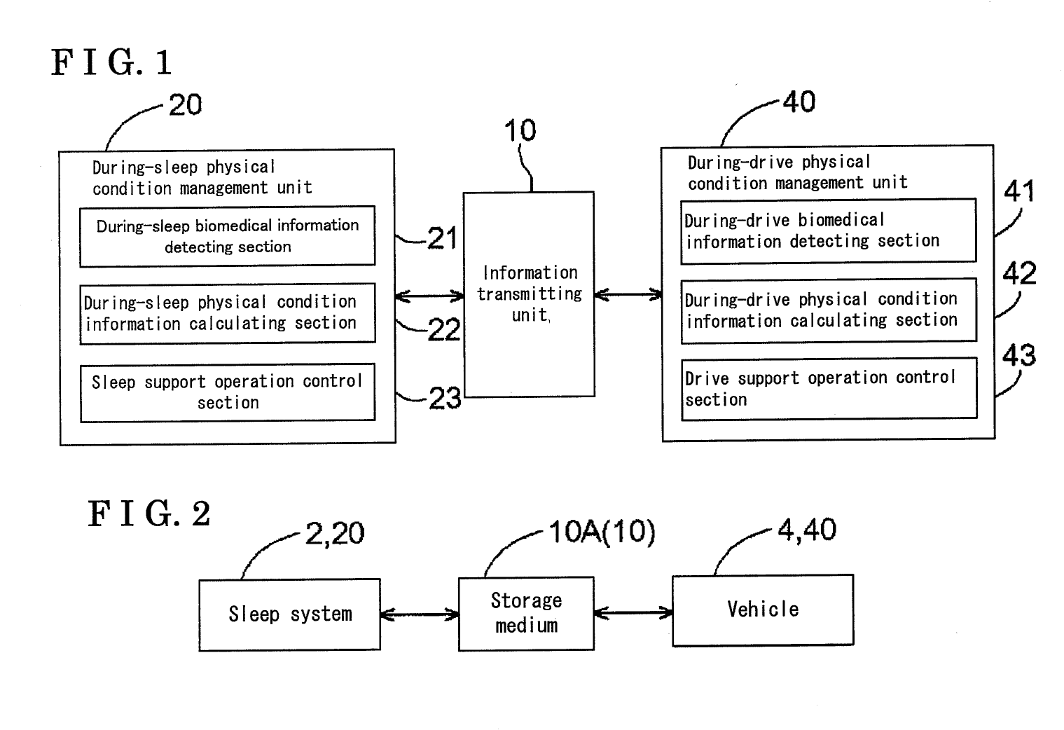

Embodiments of the present invention will be described hereunder with reference to the accompanying drawing figures. As shown in FIG. 1, a physical condition management system of the present invention includes two physical condition management units, i.e., a during-sleep physical condition management unit 20 and a during-drive physical condition management unit 40. The during-sleep physical condition management unit 20 includes a during-sleep biomedical information detecting section 21, a during-sleep physical condition information calculating section 22 and a sleep support operation control section 23. The during-drive physical condition management unit 40 includes a during-drive biomedical information detecting section 41, a during-drive physical condition information calculating section 42 and a drive support operation control section 43. The during-sleep physical condition management unit 20 and the during-drive physical condition management unit 40 transmit physical condition information and/or biomedical information therebetween via an information transmitting unit 10. The physical condition information and the biomedical information will be described later.

The during-sleep biomedical information detecting section 21 detects a during-sleep biomedical information during sleep. The during-sleep biomedical information corresponds to a biomedical information including pulse, respiration, body temperature and body motion. The sleep support operation control section 23 controls sleep support operation based on a during-sleep physical condition information calculated using the during-sleep biomedical information. The sleep support operation includes support of sleep induction, support of awakening and a recommended sleep notice. The during-drive biomedical information detecting section 41 detects a during-drive biomedical information while the user is in the vehicle. The during-drive biomedical information corresponds to the biomedical information including the pulse, the respiration and a movement of line of sight measured during driving. The drive support operation control section 43 controls drive support operation based on a during-drive physical condition information calculated using the during-drive biomedical information. The drive support operation corresponds to operation to support driving operation of the vehicle, including support of awakening the driver (user) when concentration of him or her is decreased and a recommended rest notice when a degree of fatigue of the driver (user) is increased. The during-sleep physical condition information calculating section 22 calculates the during-sleep physical condition information based on the during-sleep biomedical information and the during-drive physical condition information. The during-drive physical condition information calculating section 42 calculates the during-drive physical condition information based on the during-drive biomedical information and the during-sleep physical condition information. The information transmitting unit 10 transmits the during-sleep physical condition information and/or the during-sleep biomedical information to the during-drive physical condition information calculating section, and also transmits the during-drive physical condition information and/or the during-drive biomedical information to the during-sleep physical condition information calculating section.

FIG. 2 is a block diagram schematically showing an application example of the physical condition management system of the present invention. The during-sleep physical condition management unit 20 is provided, for example, on a sleep system 2. The during-drive physical condition management unit 40 is provided, for example on the vehicle 4.

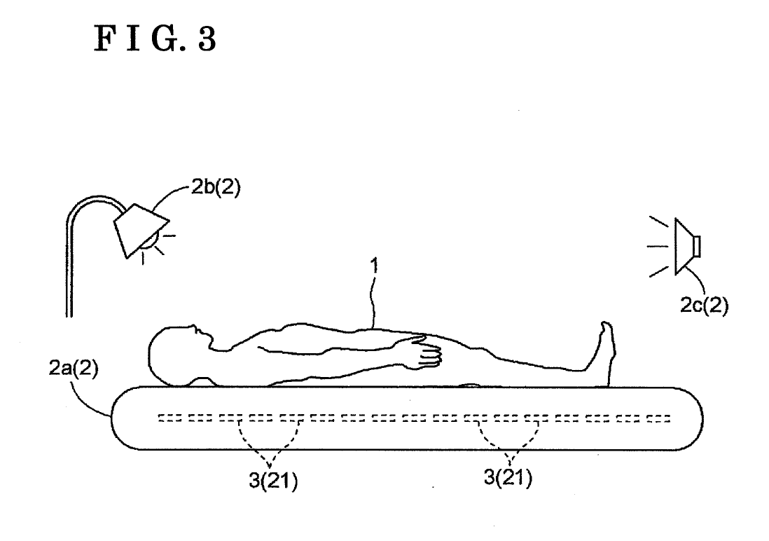

FIG. 3 is a diagram showing an example of the sleep system 2. The sleep system 2 of the present invention includes a bed 2a, a light 2b and an audio device 2c. The bed 2a may be provided with a recliner mechanism, a vibrating mechanism, a massaging mechanism and the like. The bed 2a may also be provided with an air-conditioning system (not shown), a humidifier (not shown) and the like.

The bed 2a on which a user 1 lies is provided inside thereof with a piezoelectric sensor 3. The piezoelectric sensor 3 corresponds to the during-sleep biomedical information detecting section 21 of the present invention. The piezoelectric sensor 3 detects micro vibrations due to the pulse and the respiration, and outputs a signal according to an external force. Here only the piezoelectric sensor 3 is provided as the during-sleep biomedical information detecting section 21, however, other sensors including a temperature sensor may be additionally provided. Alternatively, other sensors including a magnetic sensor may be used for detecting the pulse and respiration.

The sleep support operation control section 23 controls the light 2b and the audio device 2c of the sleep system 2. The sleep support operation control section 23 controls the sleep support operation by, for example, allowing the audio device 2c to play music in order to calm and relax the user before and during sleep or increasing brightness of the light 2b at a time of awakening. The sleep support operation control section 23 may activate the air-conditioning device and/or the humidifier. The support operation is hereinafter referred to as sleep actuation or actuation.

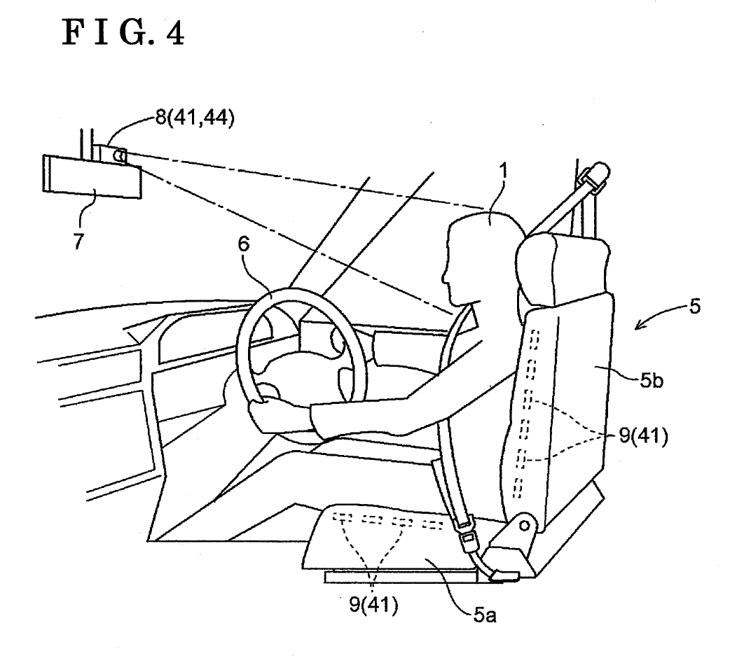

FIG. 4 shows an example of the vehicle provided with the during-drive physical condition management unit 40. A piezoelectric sensor 9 is provided on a seat 5a and/or inside a seatback 5b of a driver's seat 5 on which the user (driver) 1 is seated. The piezoelectric sensor 9 corresponds to the during-drive biomedical information detecting section 41 of the present invention. The piezoelectric sensor 9 detects the micro vibrations due to the pulse and the respiration, load, a driving posture and the like. Here, only the piezoelectric sensor 9 is provided as an example of the during-drive biomedical information detecting section 41, however, other sensors including the temperature sensor may be additionally provided. Alternatively, other sensors including the magnetic sensor may be used for detecting the pulse and respiration.

An interior rearview mirror 7 is provided with a camera 8 taking an image of the driver 1. The image taken by the camera 8 is processed in an image processing section (not shown) so that a direction of a face, a movement of a head and a movement of eyes of the driver 1 are recognized. The recognized information is a kind of biomedical information, and the camera 8 and the image processing section correspond to the during-drive biomedical information detecting section 41 of the present invention. The camera 8 may be provided on an instrument panel or a steering wheel 6, instead of on the interior rearview mirror 7.

In case that the driver's seat 5 is an electrical seat of which seat position, seat height, reclining position, headrest angle or the like is electrically adjustable, the drive support operation control section 43 controls the electrical seat in accordance with the physical condition information. In case that the seat is provided with the vibrating mechanism, the drive support operation control section 43 controls the mechanism. The drive support operation control section 43 also controls a navigation system (not shown) and an audio system (not shown). For example, to awaken the driver 1, the drive support operation control section 43 performs the drive support operation including “actuating the vibrating mechanism to vibrate the seat”, “allowing the audio system to play up-tempo music” and “allowing the navigation system to display a rest stop”. The support operation is hereinafter referred to as drive actuation or actuation.

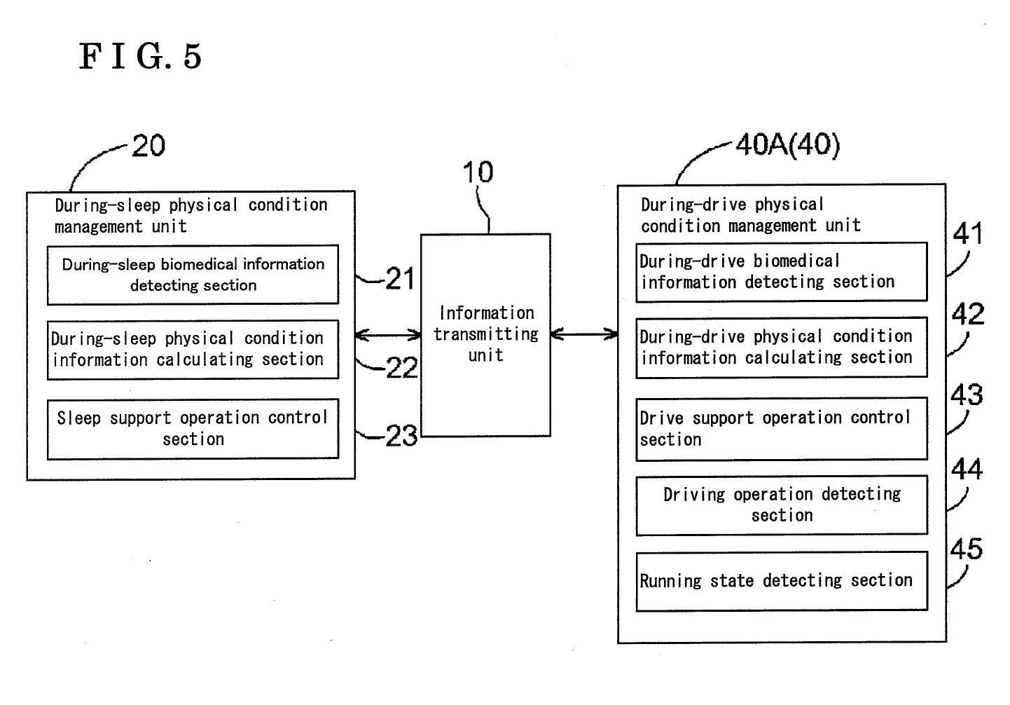

Although the user 1 is at rest in the sleep system 2, the user 1 is engaged in activities in the vehicle 4. It is preferable, therefore, that the during-drive physical condition information is calculated and the drive support operation is controlled in view of a state of driving operation and a running state of the vehicle 4. FIG. 5 is a block diagram showing an example of the physical condition management system provided with a during-drive physical condition management unit 40A which additionally includes a detecting section detecting the above-mentioned state of driving operation and the running state.

As shown in the drawing, the during-drive physical condition management unit 40A additionally includes a driving operation detecting section 44 detecting the driving operation of the vehicle 4 performed by the driver 1 and a running state detecting section 45 detecting the running state of the vehicle 4. The driving operation is detected through an operation of a gear shift lever, a steering maneuver of the steering wheel 6, the line of sight to the interior rearview mirror 7, the line of sight during visual check, an operation of an accelerator pedal, an operation of a brake pedal or the like. For example, the operation of the gear shift lever is detected by a shift position sensor (not shown) and the steering maneuver of the steering wheel 6 is detected by a steering angle sensor (not shown). The operation of the accelerator pedal and the operation of the brake pedal are detected by an accelerator sensor (not shown) and a brake sensor (not shown), respectively. In addition, the movement of the head and the line of sight of the driver 1 are detected using the image captured by the camera 8. Since the line of sight may be considered as part of the biomedical information, the camera 8 corresponds to the driving operation detecting section 44 and the during-drive biomedical information detecting section 41. The running state of the vehicle 4 is detected by use of the steering angle sensor and a speed meter (not shown). The during-drive physical condition information calculating section 42 calculates the during-drive physical condition information based on the during-drive biomedical information, the during-sleep physical condition information, the driving operation information detected by the driving operation detecting section 44, and a running state information detected by the running state detection section.

A storage medium 10A corresponds to the information transmitting unit 10 of the present invention. The storage medium 10A may be, for example, a semiconductor memory card. The sleep system 2 and the vehicle 4 are both provided with functions to read and write (rewrite) the storage medium 10A, and transmit the physical condition information therebetween via the storage medium 10A. Reading or writing is not limited to a contact method, but may be done in a non-contact method. Further, the storage medium 10A is not limited to a card that functions only as a semiconductor memory card, but may be an IC card having additional multiple functions for an electronic toll collection system, an electronic money system or the like. The storage medium 10A may also be a mobile phone or a portable audio system.

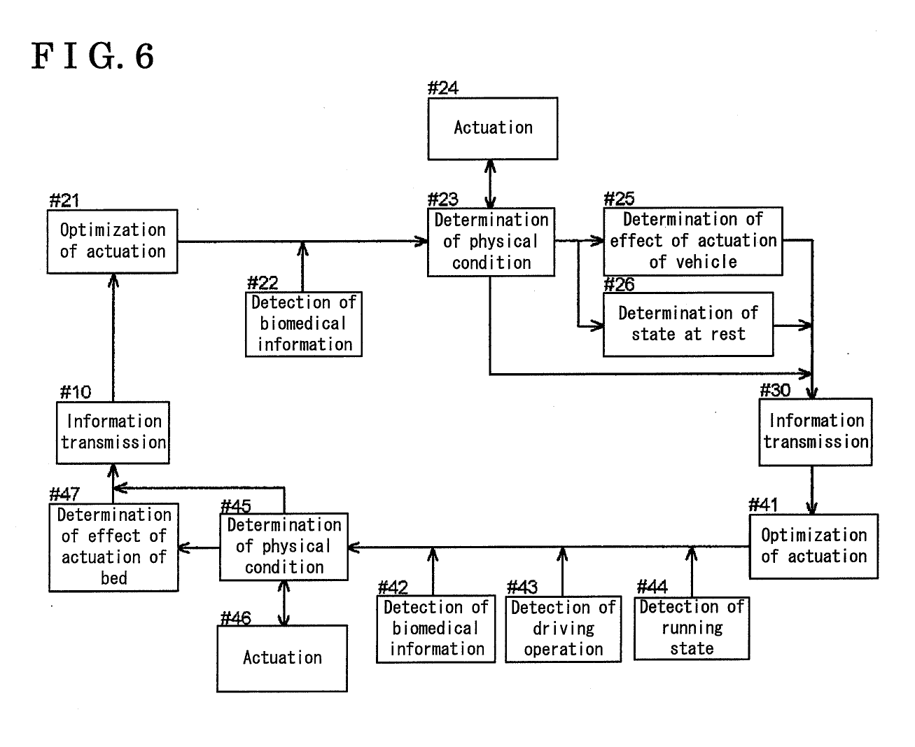

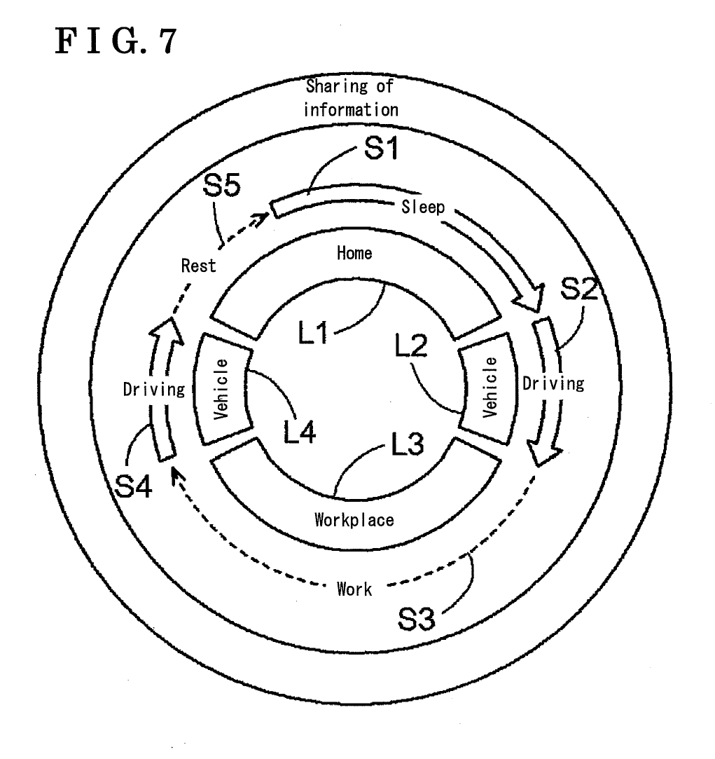

FIG. 6 is a state transition diagram of the physical condition management system of the present invention. FIG. 7 is a diagram explaining an example of a state that information is shared. A typical flow of a physical condition management will be described hereunder referring to FIGS. 6 and 7. Immediately before the user 1 goes into a sleep state, the sleep support operation control section 23 optimizes the sleep actuation according to the during-sleep physical condition information calculated in the during-sleep physical condition information calculating section 22 (#21). For example, the sleep actuation to support sleep induction is optimized. For example, sleep promotion actuation for relaxation is performed by means of massage, music, lighting and the like. Sleep induction promotion actuation is also performed, that is, application of the minor vibrations (1/f fluctuation), supply of the negative ions, regulation of the temperature and the humidity of the sleep environment, and the like. Once the user 1 goes to sleep, the biomedical information is detected by the during-sleep biomedical information detecting section 21 (#22). The during-sleep physical condition information calculating section 22 calculates the during-sleep physical condition information from the detected during-sleep biomedical information and the information (i.e., the during-drive physical condition information and/or the during-drive biomedical information) received via the information transmitting unit 10 (#23). From the physical condition information, the current physical condition is determined and the current sleep actuation is optimized (#23, #24).

The during-sleep physical condition information calculating section 22 determines, based on the during-sleep physical condition information, effects of the drive actuation performed in the vehicle 4. For example, it is determined whether or not the actuation that prevents accumulation of fatigue has been performed (#25). A state at rest is also determined from the biomedical information during sleep (#26). When the user 1 wakes up, awakening actuation is performed as the sleep actuation. For example, the brightness of the light 2b is gradually increased to bring the user 1 to a state of a light sleep in consideration of a REM sleep time, and acoustic effect is provided by the audio device 2c for eventually awakening the user 1 in a refreshed state. The during-sleep physical condition includes the physical condition at the time of awakening. The during-sleep biomedical information and/or the during-sleep physical condition information that is calculated in the during-sleep physical condition information calculating section 22 are transmitted to the during-drive physical condition management unit 40 via the information transmitting unit 10 (#30).

When the during-drive physical condition management unit 40 receives the during-sleep physical condition information, the during-drive physical condition information is calculated in the during-drive physical condition information calculating section 42. The drive support operation control section 43 optimizes the drive actuation according to the during-drive physical condition information (#41). When the during-drive physical condition information calculating section 42 determines, from the during-sleep physical condition information, that the user 1 is suffering from sleep deprivation, or the like, the during-drive physical condition information calculating section 42 allows the awakening actuation to be performed at an early stage via the drive support operation control section 43. For example, the drive support operation control section 43 performs the drive actuation including “allowing the audio system to play up-tempo music” and “allowing the navigation system to display a rest stop”. Alternatively, the drive support operation control section 43 may allow the vibrating mechanism to operate with short intervals (for example, one-minute interval).

During driving, the biomedical information, the driving operation and the running state are detected by the during-drive biomedical information detecting section 41, the driving operation detecting section 44 and the running state detecting section 45 and the like. The during-drive physical condition information calculating section 42 calculates the during-drive physical condition information from the during-drive biomedical information, the driving operation and the running state that are detected, and the information received via the information transmitting unit 10 (i.e., the during-sleep physical condition information and/or the during-sleep biomedical information) (#45). From the physical condition information, the current physical condition is determined and the current actuation is optimized (#45, #46).

The during-drive physical condition information calculating section 42 determines, from the during-drive physical condition information, effects of the actuation performed by the sleep system 2. For example, the during-drive physical condition information calculating section 42 determines whether or not the sleep actuation that provides good sleep and smooth awakening has been performed (#47). The during-drive physical condition information calculating section 42 also determines a state during activity (for example, a state of excitement) based on the biomedical information during drive (#45). The during-drive biomedical information and/or the during-drive physical condition information that is calculated in the during-drive physical condition information calculating section 42 are transmitted to the during-sleep physical condition management unit 20 via the information transmitting unit 10 after the user 1 returns home (#10).

FIG. 7 is a diagram explaining an example of the state that information is shared between the during-sleep physical condition management unit 20 and the during-drive physical condition management unit 40. The example shows a cycle of sleeping (S1) at home L1, driving (S2) the vehicle L2, working (S3) at workplace L3, driving (S4) the vehicle L4, resting (S5) and sleeping (S1) at home L1. As described above, the physical condition information during sleep (S1) is used during drive (S2). Results of determination made during drive (S2) on the effects of the actuation during sleep (S1) is transmitted as one of the during-drive physical condition information after driving (S4) home, and then used during the next sleep (S1). The physical condition information obtained during driving (S4) home is used in the actuation for the rest (S5) and for the next sleep (S1). As the actuation, for example, the physical condition management system may allow the audio device 2c to play appropriate BGM when the user is at rest, promote sleep and provide message.

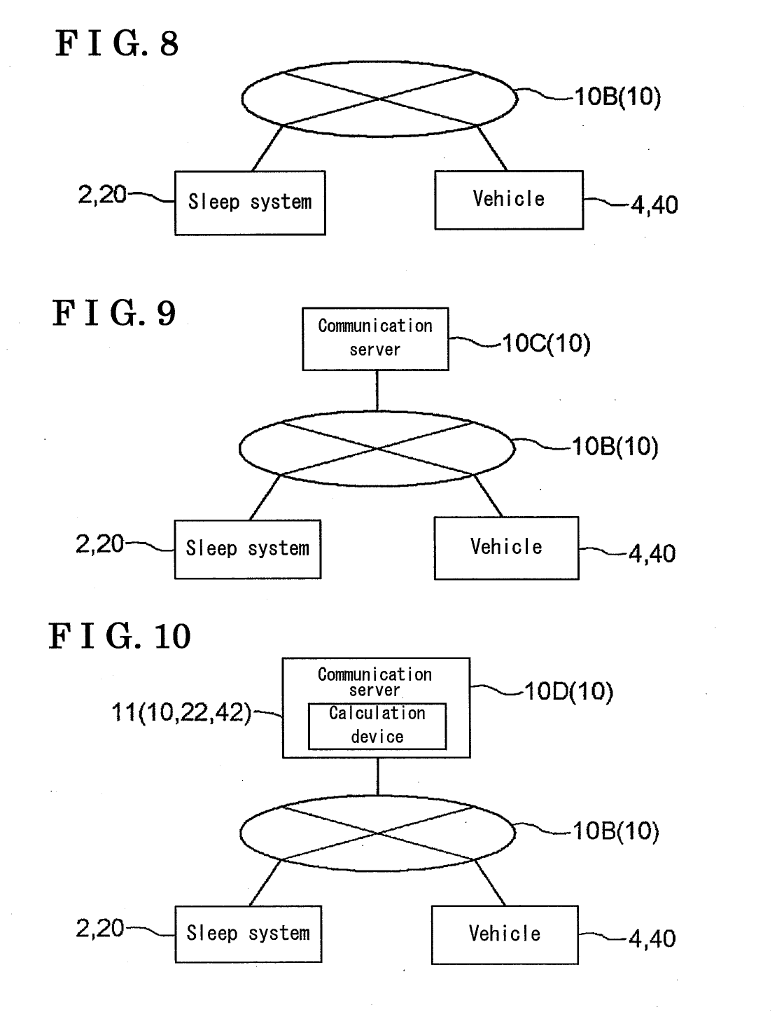

FIGS. 8 to 10 are block diagrams schematically showing other examples of the physical condition management system of the present invention. As shown in FIG. 8, the information transmitting unit 10 may be constituted by a network 10B such as the Internet. This structure allows the information to be conveniently transmitted by eliminating trouble of carrying the storage medium 10A. As shown in FIG. 9, the information transmitting unit 10 may be constituted by the network 10B and a communication server 10C. In the structure shown in FIG. 8, the sleep system 2 and the vehicle 4 need to be always communicable with each other. In the structure shown in FIG. 9, however, the transmitted data is stored in the communication server 10C, which enables a receiver to access to the information as needed.

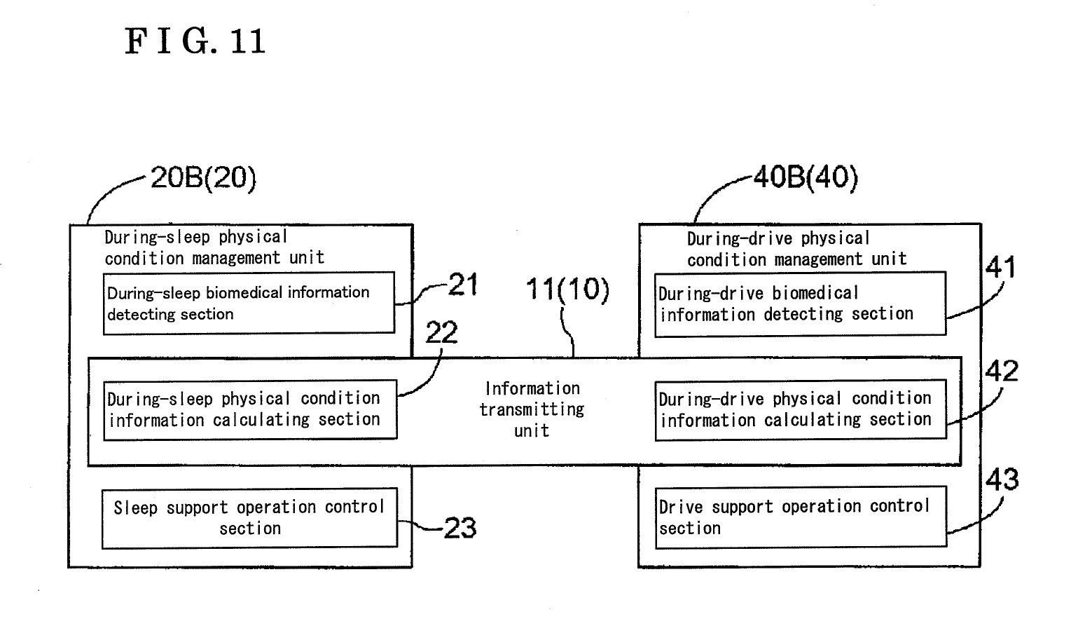

Further, as shown in FIG. 10, a communication server 10D may include therein a calculation device 11. As shown in FIG. 11, the calculation device 11 serves at least as part of the during-sleep physical condition information calculating section 22 and the during-drive physical condition information calculating section 42. This structure enables the information shared between a during-sleep physical condition management unit 20B and a during-drive physical condition management unit 40B to be managed in an integrated manner.

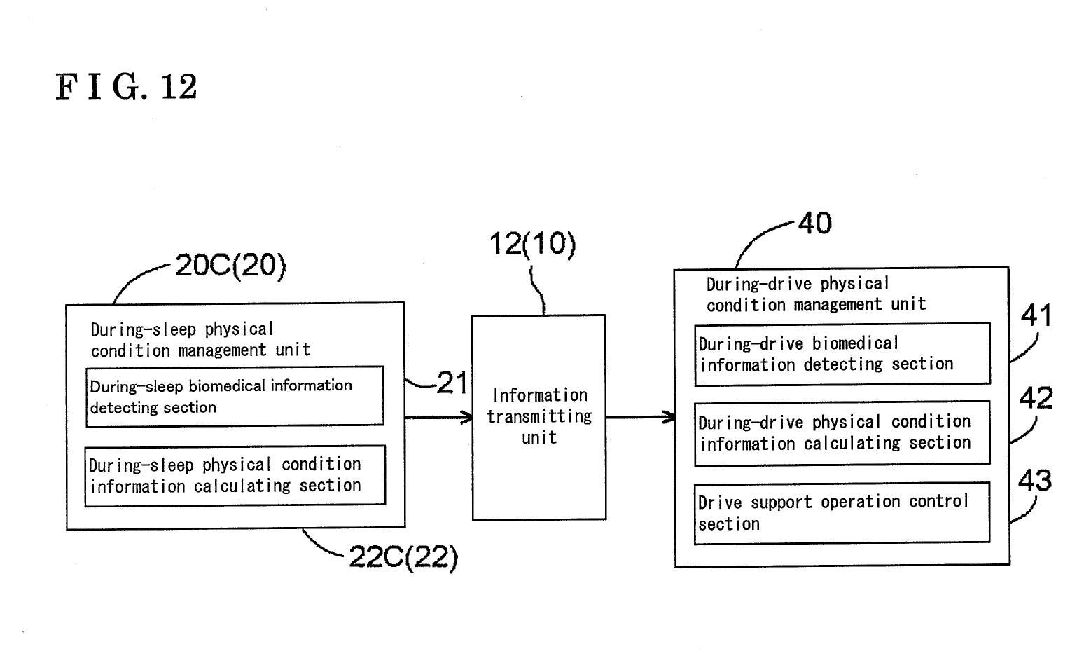

In the embodiment described above, the during-sleep physical condition management unit 20 and the during-drive physical condition management unit 40 transmit the information therebetween. However, if the during-sleep physical condition information and/or the during-sleep biomedical information are available for use in the during-drive physical condition management unit 40, beneficial effects in improving the control of the drive support operation are consistently achieved. Therefore, a system structure may be applied in which at least the during-sleep physical condition information and/or the during-sleep biomedical information are transmitted to the during-drive physical condition management unit 40. FIG. 12 is a block diagram schematically showing an example of such structure of the physical condition management system.

As shown in the drawing, the physical condition management system of this example includes a during-sleep physical condition management unit 20C, the during-drive physical condition management unit 40 and an information transmitting unit 12. Unlike the physical condition management systems described in the earlier examples, the information transmitting unit 12 transmits the during-sleep physical condition information and/or the during-sleep biomedical information to the during-drive physical condition information calculating section. That is, the during-drive physical condition information or the during-drive biomedical information is not necessarily transmitted to a during-sleep physical condition information calculating section 22C in this example. The during-sleep physical condition management unit 20C includes the during-sleep biomedical information detecting section 21 and the during-sleep physical condition information calculating section 22C. In this example, the sleep support operation or the related control section is not necessary. The during-drive physical condition management unit 40 has the same structure as described above. The during-sleep biomedical information detecting section 21 of the during-sleep physical condition management unit 20C detects the during-sleep biomedical information in the same manner as described above. The during-sleep physical condition information calculating section 22C calculates the during-sleep physical condition information based on the during-sleep biomedical information. As described above, in this example, the information transmitting unit 12 does not transmit the during-drive physical condition information or the during-drive biomedical information to the during-sleep physical condition information calculating section 22C. Consequently, the during-sleep physical condition information calculating section 22C calculates the during-sleep physical condition information based on the during-sleep biomedical information without considering the during-drive physical condition information.

As described above, the present invention provides the physical condition management system that manages the physical condition based on the biomedical information obtained before and while the person is in the vehicle without bothering him or her.

### INDUSTRIAL APPLICABILITY

The present invention is applicable to a physical condition management system which manages a physical condition of a person by obtaining his or her biomedical information. For example, the present invention is applicable to a sleep system constructed in a building and having a bed as a core of the system, a drive management system mounted on the vehicle for managing a condition of a driver and a physical condition management system of an individual person where the sleep system and the drive management system are integrated.

1. A physical condition management system for managing a physical condition of a person by obtaining biomedical information, comprising:
a during-sleep biomedical information detecting section detecting a during-sleep biomedical information during sleep;
a sleep support operation control section controlling a sleep support operation supporting at least sleep induction based on a during-sleep physical condition information calculated by use of the during-sleep biomedical information;
a during-drive biomedical information detecting section detecting a during-drive biomedical information while a user is a vehicle;
a drive support operation control section controlling a drive support operation supporting at least a driving operation of the vehicle based on a during-drive physical condition information calculated by use of the during-drive biomedical information;
a during-sleep physical condition information calculating section calculating the during-sleep physical condition information based on either one or both of the during-drive physical condition information and the during-drive biomedical information, and the during-sleep biomedical information;
a during-drive physical condition information calculating section calculating the during-drive physical condition information based on either one or both of the during-sleep physical condition information and the during-sleep biomedical information, and the during-drive biomedical information; and
an information transmitting unit transmitting either one or both of the during-sleep physical condition information and the during-sleep biomedical information to the during-drive physical condition information calculating section, and transmitting either one or both of the during-drive physical condition information and the during-drive biomedical information to the during-sleep physical condition information calculating section.

2. The physical condition management system as set forth in. claim 1, wherein the during-drive physical condition information calculating section calculates the during-drive physical condition information including at least either one or both of a degree of concentration on driving and a degree of fatigue from driving based on the during-drive biomedical information, and the during-sleep physical condition information including at least either one or both of a quality of sleep and a degree of wakefulness at a time of awakening, and
the drive support operation control section operates the drive support operation including at least either one or both of an awakening operation and a rest induction operation based on the during-drive physical condition information.

3. The physical condition management system. as set forth in claim 1, wherein the during-sleep physical condition information calculating section calculates the during-sleep physical condition information including at least a required quality of sleep based on the during-sleep biomedical information, and the during-drive physical condition information including at least either one or both of a degree of excitement by driving and cumulative fatigue from driving, and the sleep support operation control section operates the sleep support operation including at least either one or both of a sleep promotion operation and a sleep induction operation based on the during-sleep physical condition information.

4. The physical condition management system as set forth in claim 1, further comprising:
a driving operation detecting section detecting a driving operation of the vehicle; and a running state detecting section detecting a running state of the vehicle, wherein the during-drive physical condition information calculating section calculates the during-drive physical condition information based on the during-drive biomedical information, the during-sleep physical condition information, a driving operation information detected by the driving operation detecting section and a running state information detected by the running state detecting section.

5. The physical condition management system as set forth in claim 1, wherein the information transmitting unit transmits the during-sleep physical condition information and the during-drive physical condition information by means of a communication medium or a rewritable storage medium.

6. A physical condition management system for managing a physical condition of a person by obtaining a biomedical information, comprising:
a during-sleep biomedical information detecting section detecting a during-sleep biomedical information during sleep;
a during-sleep physical condition information calculating section calculating a during-sleep physical condition information based on the during-sleep biomedical information;
a during-drive biomedical information detecting section detecting a during-drive biomedical information while a user is in a vehicle;
a drive support operation control section controlling a drive support operation supporting at least a driving operation of the vehicle based on a during-drive physical condition information calculated by use of the during-drive biomedical information;
a during-drive physical condition information calculating section calculating the during-drive physical condition information based on either one or both of the during-sleep physical condition information and the during-sleep biomedical information, and the during-drive biomedical information; and
an information transmitting unit transmitting either one or both of the during-sleep physical condition information and the during-sleep biomedical information to the during-drive physical condition information calculating section.

7. The physical condition management system as set forth in claim 1, wherein the during-sleep biomedical information is selected from among pulse, respiration, body temperature and body motion measured during sleep.

8. The physical condition management system as set forth in claim 1, wherein the sleep support operation includes a support operation of sleep induction, a support operation of awakening and/or a recommended sleep notice.

9. The physical condition management system as set forth in claim 1, wherein the drive support operation includes the support operation of awakening provided upon a decline of a degree of a driver's concentration on driving and/or a recommended rest notice provided in case of fatigue.
